(19) United States
(12) Patent Application Publication
(10) Pub. No.: US 2012/0164111 A1
(43) Pub. Date: Jun. 28, 2012

(54) NOVEL POST-TRANSLATIONAL FIBRINOGEN VARIANTS

(76) Inventor: Kurt Osther, Scottsdale, AZ (US)

(21) Appl. No.: 13/264,489

(22) PCT Filed: Apr. 13, 2010
(86) PCT No.: PCT/EP2010/054834

§ 371 Date: Feb. 28, 2012

Related U.S. Application Data
(60) Provisional application No. 61/212,667, filed on Apr. 14, 2009.

(51) Int. Cl. A61K 38/36; A61P 7/00; A61K 38/48; A61K 35/12; C12P 21/00; C07K 14/75

(52) U.S. Cl. 424/93.7; 435/69.6; 530/382; 514/13.5; 424/94.64

(57) ABSTRACT

Provided are novel post-translationally modified variants of fibrinogen, in particular human fibrinogen, a method for producing and isolating said variants, as well as methods of using the variant, in particular in systemic and local/topical treatment and prophylaxis of excessive bleeding.

### FIELD OF THE INVENTION

The present invention relates to the field of molecular biology and in particular to the provision of novel post-translational variants of human fibrinogen prepared by recombinant means. The invention also pertains to methods and uses relevant to these novel variants.

### BACKGROUND OF THE INVENTION

Fibrinogen, one of the critical structural plasma proteins, is a 340-kDa glycoprotein as described by Doolittle et al. (Doolittle R F, Spraggon G, Everse S J. Three-dimensional structural studies on fragments of fibrinogen and fibrin. Curr Opin Struct Biol. (1998) 8:792-798). Vertebrate fibrinogen molecules are composed of three different pairs of chains (α2, β2 and γ2 chains), where the two members of a pair are identical. For human fibrinogen, the reduced Aα-, Bβ-, and γ chains have molecular weights of approximately 65,000, 55,000, and 47,000.

Fibrinogen-Related domains (FReDs); C-Terminal Globular Domain of Fibrinogen

Fibrinogen is involved in blood clotting, being activated by thrombin to assemble into fibrin clots. The N-termini of 2×3 chains associate to form a globular arrangement called the disulfide knot. The C-termini of the fibrinogen chains end in globular domains, which are not completely equivalent. The C-terminal globular domains of the γ chains (C-gamma) dimerize and bind to the GPR motif of the N-terminal domain of the α chain, while the GHR motif of N-terminal domain of the β chain binds to the C terminal globular domains of another β chain (C-beta), thus leading to lattice formation. Following vascular injury, fibrinogen is cleaved by thrombin to form fibrin which is the most abundant component of blood clots. In addition, various cleavage products of fibrinogen and fibrin regulate cell adhesion and spreading, display vasoconstrictor and chemotactic activities, and are mitogens for several cell types. Previously described mutations in one or more of the fibrinogen genes lead to several disorders, including afibrinogenemia, dysfibrinogenemia, hypodysfibrinogenemia and thrombotic tendency.

It is known that there are several variances in fibrinogen in humans depending on genetic factors: it has been suggested that as much as 51% of the variance in plasma fibrinogen levels may be accounted for by genetic factors. Discrepancies within or between the fibrinogen protein sequence data and early coding region DNA sequence data generated by several laboratories has been suggested to be the result of experimental artifacts, rare or new variants, or to indicate true, established polymorphisms.

Colafranceschi et al. have studied he hydrophobicity pattern distribution in the Aα-, Bβ- and γ-chains of human fibrinogen using a nonlinear method, recurrence quantification analysis, in the wild type and in a number of naturally occurring or artificial mutants from the point of view to find a structural basis for distinguishing between silent and pathological mutants (Colafranceschi M, Papi M, Giuliani A, Amiconi G, Colosimo A., Pathophysiol. Haemost. Thromb., 2006, 35:417-27). Colafranceschi et al. were successful in the case of mutations on the Aα-chain, thanks to the peculiar features of this chain as compared to the other two. Relevant findings concerning the point mutants of the Aα-chain are the following: (a) the recurrence quantification analysis-based classification of such mutants is in good agreement with the clinical classification, and (b) the location of the mutated residue on the sequence plays a more relevant role than its hydrophobic features. According to Colafranceschi et al. artificial point mutants in the terminal zone (600-866 residues) of the extended isoform of the Aα-chain are clustered together with the natural hemorrhagic mutants of the first (1-207) residues.

Ajjan et al. describes a common variation in the C-terminal region of the fibrinogen Bβ chain. Fibrinogen Bβ Arg448Lys is according to Ajjan and others, the result of a common polymorphism, positioned within the carboxyl terminus of the Bβ-chain of the molecule (Ajjan R, Lim B C B, Standeven K F, Harrand R, Dolling S, Phoenix F, Greaves R, Abou-Saleh R H, Connell S, Smith Dam, Weisel J W, Grant P J, Ariëns R A S. Blood, (2008) 111:643-649; Baumann R E, Henschen A H, Blood (1993) 82:2117-2124; Carter A M, Catto A J, Bamford J M, Grant P J. Arterioscler. Thromb. Vasc. Biol. (1997), 17:589-594).

Recent progress has been made in understanding the molecular basis of congenital afibrinogenemia, an autosomal recessive coagulation disorder characterized by the complete absence of detectable fibrinogen. The first causative mutations were identified for this disorder in a non-consanguineous Swiss family; these were homozygous deletions of approximately 11 kb of the fibrinogen alpha chain (FGA α) encoding gene. Haplotype data implied that the deletions occurred on distinct ancestral chromosomes, suggesting that this region of the FGA gene may be susceptible to deletion by a common mechanism. All the deletions were identical to the base pair, and probably resulted from non-homologous (illegitimate) recombination. In a subsequent study of 13 unrelated patients with congenital afibrinogenemia the authors analyzed the FGA gene in order to identify the causative mutations, and to determine the prevalence of the 11-kb FGA deletion. Three frame-shift mutations, two nonsense mutations, and one other splice site mutation were also characterized. Other studies identified one further FGAα nonsense mutation, two FGBβ miss-sense mutations, and one FGγ nonsense mutation, all in homozygosis in a single patient (Neerman-Arbez M., Ann N Y Acad Sci. 2001;936:496-508).

In conclusion, the majority of patients have truncating mutations in the FGAα gene although, intuitively, all three fibrinogen genes could be predicted to be equally implicated. The existing knowledge will facilitate molecular diagnosis of the disorder, permit prenatal diagnosis for families who so desire, and pave the way for new therapeutic approaches such as gene therapy.

Previous genotype-phenotype association studies of fibrinogen have been limited by incomplete knowledge of genomic sequence variation in FGEβ, FGAα, and FGγ within and between major ethnic groups. The linkage disequilibrium is characterized as patterns and haplotype structure across the human fibrinogen gene locus in European- and African-American populations. In young adults, fibrinogen multi-locus genotypes appeared to be associated with plasma fibrinogen levels. The specific single nucleotide polymorphism and haplotype patterns for these associations appears to differ according to population and also according to phenotypic assay. It seems likely that a substantial proportion of the heritable component of plasma fibrinogen concentration is due to genetic variation outside the three fibrinogen genes.

Confounding causes and reverse causation have been proposed as explanations for the association between high fibrinogen levels and cardiovascular disease. Genetic variants can alter fibrinogen characteristics and are not subject to these problems. To determine the fibrinogen plasma levels for genotypic variants in fibrinogen-Aα (FGAα Thr312Ala) and fibrinogen-Bβ (FGBβ-455G/A), and whether these variants are associated with arterial thrombosis fibrinogen genotypes were determined in a population-based case-control study including women aged 18-50 years. The FGBβ-455G/A variant increased plasma fibrinogen levels, whereas the FGAα Thr312Ala variant lowered plasma fibrinogen levels, albeit to a modest extent. The risk of ischemic stroke was altered when the homozygote minor allele was compared with the homozygote major allele. The FGAα Thr312Ala single-nucleotide polymorphism (SNP) was associated with a decrease in risk, whereas the FGBβ-455G/A SNP might have increased the risk. The risk of myocardial infarction was not altered for either SNP. With the genetic variations as markers of plasma fibrinogen levels alterations, thereby ruling out confounding and reverse causation, the results suggested were that plasma fibrinogen levels could play a more pronounced role as risk factors for ischemic stroke than for myocardial infarction (Siegerink B, Rosendaal F R, Algra A. J Thromb Haemost. (2009) 7:385-90).

Factor XIII and fibrinogen are unusual among clotting factors in that neither is a serine protease. Fibrin is the main protein constituent of the blood clot, which is stabilized by factor XIIIa through an amide or isopeptide bond that ligates adjacent fibrin monomers. Many of the structural and functional features of factor XIII and fibrin(ogen) have been elucidated by protein and gene analysis, site-directed mutagenesis, and x-ray crystallography. However, some of the molecular aspects involved in the complex processes of insoluble fibrin formation in vivo and in vitro remain unresolved. The findings of a relationship between fibrinogen, factor XIII, and cardiovascular or other thrombotic disorders have focused much attention on these 2 proteins.

Of particular interest are associations between common variations in the genes of factor XIII and altered risk profiles for thrombosis. Although there is much debate regarding these observations, the implications for the understanding of clot formation and therapeutic intervention may be of major importance.

A common polymorphism Val to Leu at position 34 in the FXIII A subunit has been investigated as a risk determinant of thrombosis. Because Val34Leu is considered close to the thrombin cleavage site, the hypothesis is that it would alter the function of FXIII: Analysis of FXIII subunit proteolysis by thrombin using sodium dodecyl sulfate-polyacrylamide gel electrophoresis and high-performance liquid chromatography has demonstrated that FXIII 34Leu is cleaved by thrombin more rapidly and at lower doses than 34Val. Mass spectrometry of isolated activation peptides confirmed the predicted single methyl group difference and demonstrated that the thrombin cleavage site is unaltered by Val34Leu. Kinetic analysis of activation peptide release demonstrated that the catalytic efficiency (k(cat)/K(m)) of thrombin was 0.5 for FXIII 34Leu and 0.2 (micromol/L)(−1)×sec(−1) for 34Val. Presence of fibrin increased the catalytic efficiency to 4.8 and 2.2 (micromol/L)(−1)×sec(−1), respectively. Although the 34Leu peptide is released at a similar rate as fibrinopeptide A, the 34Val peptide is released more slowly than fibrinopeptide A but more quickly than fibrinopeptide B generation. Cross-linking of gamma- and alpha-chains appeared earlier when fibrin was incubated with FXIII 34Leu than when with 34Val.

Fully activated 34Leu and 34Val FXIII exhibit similar cross-linking activities. Analysis of fibrin clots prepared using plasma from FXIII 34Leu subjects by turbidity and permeability measurements has shown reduced fiber mass/length ratio and porosity compared to 34Val. The structural differences have been confirmed by electron microscopy. These results demonstrated that Val34Leu accelerates activation of FXIII by thrombin and consequently appear to affect the structure of the cross-linked fibrin clot in this manner.

In U.S. Pat. No. 6,037,457, a recombinant fibrinogen is produced in Chinese Hamster Ovary (CHO) cells. Illustrative conditions under which transfected mammalian cells can be cultured to express fibrinogen are provided: cells are expanded in roller bottles with adherent microcarrier beads in serum containing medium for a period of 4-5 weeks, followed by cell culturing in a total of 200 ml of serum-free medium (Dulbecco's Modified Eagle's Medium/F12 medium containing 10 IU penicillin/ml, 10 mg streptomycin/ml, 10 U aprotinin/ml, and 10 μg/ml each of insulin, sodium selenite, and transferrin) at 37° C. for three weeks prior to commencing the collection of conditioned culture medium every four to seven days in order to start a purification process wherein, according to the authors of U.S. Pat. No. 6,037,457, this is in many cases initiated with an ammonium sulphate precipitation.

### DESCRIPTION OF THE INVENTION

U.S. Pat. No. 6,037,457 exclusively deals with provision of recombinant fibrinogen, and furthermore, does not solve the problem in regards of providing a complete pure recombinant human post translated system as described WO 2007/103447. U.S. Pat. No. 6,037,457 hence does not relate to any findings with respect to post-translational differences between fibrinogens obtained, nor to the presence of hitherto undisclosed post-translational species of fibrinogen.

The present invention relates to a novel concept and method for preparing recombinant human fibrinogen, which in particular pertains to utilization of hitherto unknown identifiable changes in the non-reduced biologically active Aα, Bβ, γ chains of human fibrinogen as a consequence of the recombinant expression system selected for production of these fibrinogen chains. It was surprisingly observed that fibrinogen produced by HEK cells disassociated into at least two different fibrinogen compositions, where the fibrinogens exhibited significant differences in thrombin-activated polymerization.

According to the present invention there is hence provided two individual fibrinogens when the alpha, beta and gamma fibrinogen chain encoding genes are built into promoter and vector systems and transfected into certain human cell lines, whereafter the expression products from these cells are harvested and isolated.

Until now the inventor has presently succeeded in establishing a transfected human cell system capable of expressing these novel differences in the fibrinogen capable, by certain purification methods to produce at least 2 (two) different distinguishable recombinant human fibrinogen, termed F5 and F6 herein.

Hence, in a first aspect, the present invention hence provides for a method for preparing fibrinogen comprising:

providing cells transfected with expression vectors encoding individual fibrinogen chains, subsequently culturing said cells under conditions that facilitate production by said cells of fibrinogen, and subsequently recovering said fibrinogen and separating said fibrinogen in at least two post-translationally modified species distinguished by differing migration patterns in a PAGE gel and/or by differing polymerization upon activation with thrombin. In a preferred embodiment, the fibrinogen obtained is human fibrinogen or a human fibrinogen including up to 10 amino acid mutations in each fibrinogen chain. In particular, each of the α, β and γ chains may independently comprise 0, 1, 2, 3, 4, 5, 6, 7, 8, 9, or 10 single amino acid mutations.

Superior results have been obtained using human cells for the preparation, so it is preferred that the transfected cells used in the method of the first aspect are constituted by human cells, e.g. selected from a human cell clone or a human cell line. Especially preferred cells are HEK cells, such as the HEK293t and HEK293ts cells obtainable from HumanZyme, 2201 West Campbell Park Drive Suite 24, Chicago, Ill. 60612, USA.

As will be clear from the present examples, the method of the invention has resulted in separation of two species of recombinant human fibrinogen expressed from the same set of chain encoding vectors in one type of cells. In the first aspect of the invention it is hence preferred that the method entails that human fibrinogen is separated into at least two post-translationally modified forms, wherein one (F5) has a PAGE migration pattern and polymerization characteristics similar to serum fibrinogen and wherein another (F6) has a PAGE migration pattern, which differs in band intensity, but has polymerization characteristics similar to fibrinogen expressed by CHO cells. Further distinguishing features between the F5 and F6 fibrinogens are that the a chain in reduced fibrinogen (i.e. fibrinogen, where disulfide bridges have been removed by addition of a reducing agent), have different properties: in PAGE, the F6 band is distinct and narrow, whereas the F5 band appear less distinct and more fuzzy (implying that the F5 protein has a greater intramolecular variation in charged groups than F6).

It is also found that recombinant Fibrinogen F5 expressed by human cells is authentic-like compared to plasma-derived fibrinogen, and more authentic-like than F6, as can be seen on the PAGE gels (FIG. 4) and also more authentic-like than CHO cell derived Fibrinogen. However, recombinant Fibrinogen F6 also differs from CHO cell derived Fibrinogen on PAGE gels. The recombinant fibrinogen F6 appears as a stronger and more dense and distinct band in non-reduced form, compared to the broader and more diffuse band observed on Fibrinogen F5 and on CHO derived Fibrinogen. Also the alpha chain in the reduced fibrinogen F6 appears to be at a relatively higher concentration compared to both Fibrinogen F5 and CHO cell derived fibrinogen. Finally, the individual polymerization patterns after having been induced (activated) by thrombin points to F5 being more authentic-like than F6 and CHO cell derived fibrinogen.

So, it appears that recombinant human fibrinogen produced in human cells (and as described in this invention) contains at least two distinct fibrinogen post-translational (PT) species. As used herein, a “fibrinogen PT species” denotes a post-translational variant of a protein, which has the exact same amino acid composition as another fibrinogen molecule; the two species merely differ in their molecules due to post-translational modifications, such as glycosylation, lipidation, phosphorylation etc.

These at least two different fibrinogen protein species are identified when harvested and can be isolated as individual species, e.g. by employing the purification methods described herein. This enables recombinant production of fibrinogen, separation in different PT species and consequently provision of fibrinogen preparations containing balanced amounts of the the at least 2 different PT species, hence enabling production of human fibrinogen compositions having specified and reproducible polymerization characteristics.

Hence, in a second aspect of the invention is provided a substantially pure preparation of human fibrinogen, which is free from non-fibrinogen serum protein and where the fibrinogen consists of:

a) F5 fibrinogen,

b) F6 fibrinogen, or

c) a mixture of F5 and F6 fibrinogen.

It is contemplated that as is the case for recombinantly produced fibrinogen exemplified herein, such multi-PT species production will occur in other transfected cells as well as in cloned or transgenic animals, so that fibrinogens obtained from such transfected cells in reality is a composition of different fibrinogen PT species—this is hence also an aspect of the invention. Alternatively, the invention provides for mixtures of at least two fibrinogen PT species, where different PT species of fibrinogen are obtained from two or more different cell types.

The differences between F5 and F6 is not dealt with in the above-discussed abnormal conditions related to pathological genetic changes in fibrinogen, and it does not appear that the prior art has anticipated the possibility of a distribution of different post-translational forms of fibrinogen in normal plasma. However, based on the present findings, it is conceivable that such different form are indeed present in plasma in nature, meaning that plasma harbours a heterogeneity of fibrinogens not previously described. One of these probable plasma fibrinogens (such as F6) may be more or less inactivated during the purification process applied on plasma fibrinogen or one of the PT fibrinogen species may be more susceptible to heat inactivation often applied to plasma fibrinogen in order to inactivate pathogens.

In line with the first aspect of the invention which provides for production of human fibrinogens having up to 10 amino acid changes in each fibrinogen chain, the preparation of the present invention also includes those where the human fibrinogen has up to 10 amino acid changes, independently in each chain (i.e. 1, 2, 3, 4, 5, 6, 7, 8, 9 or 10 single amino acid changes in each of the α, β and γ chains). So, also part of the invention is a fibrinogen comprising at least one fibrinogen chain comprising an amino acid shift. For instance, a proline to alanine shift at e.g. position 162 in the beta chain.

The initial recombinant Fibrinogen prepared in the work leading to the present invention contained a proline residue in position 162 instead of an alanine in the beta chain. The resulting “initial” fibrinogen was found, when tested by an independent laboratory, to be significantly slower in polymerizing than an “internal golden standard” (Calbiochem human plasma fibrinogen) used as control. When the same laboratory later tested the second recombinant human fibrinogen prepared with a beta chain where we had removed the proline and entered alanine in position 162 and expressed this in transfected in HEK293t cells, it was found that recombinant human fibrinogen with alanine instead of proline at position 162 in the beta chain) exhibited at least the same speed of polymerization as the “golden standard” Calbiochem human plasma fibrinogen. It is, however, clear that this change in the Bβ chain may not necessarily be the only adjustment that would result in provision of the F5 and F6 fibrinogens, other changes both in the Bβ, the Aα and γ may provide a basis for developing F5 and F6 fibrinogens or recombinant F5 and F6 fibrinogens.

However, a preferred fibrinogen of the invention comprises a non-proline in position 162 of the beta-chain, such as L-alanine, L-cysteine, L-aspartic acid, L-glutamic acid, L-phenylalanine, glycine, L-histidine, L-isoleucine, L-lysine, L-leucine, L-methionine, L-asparagine, L-glutamine, L-arginine, L-serine, L-threonine, L-valine, L-tryptophan, and L-tyrosine in this position.

Recombinant F5 and F6 human fibrinogens may be prepared in human cells ranging from HEK cells (as demonstrated herein), over PER-C6 cells or derivates thereof. Also any suitable human stem cell or human precursor cell may be transfected with Aα, Bβ, and γ fibrinogen gene whether built into a promoter and vector system individually or all of the three genes in the same promoter and vector system, with any other changes in the three individual genes constituting the fibrinogen genes.

It appears among other things that non-reduced fibrinogens can be characterized by their behavior in PAGE gels and by their particular pattern of Aα, Bβ, and γ chains under reduced conditions. It is particularly noteworthy that differences appear in the Aα protein chain in the F5 and F6 recombinant human fibrinogens. The Aα chain was indeed also stronger on the PAGE gels when compared to the CHO derived Fibrinogen. Various ratios of α,β,γ, were tested because initial studies revealed that the β chain may be the limiting factor to obtain even consistently intact fibrinogen, instead of obtaining alpha-gamma “inactive fibrinogen” (having no evidence of the presence of a beta chain). Therefore ratios 1:1:1, 1:2:1 and 1:5:1 were tested, so as to provide variations in the amount of available beta chain, compared to alpha and gamma chains.

It is therefore anticipated in this invention that an isolation of one of the genes, namely the Aα gene enables mixing the F5 and F6 genes (or expression products thereof) and using the relative mix of the α, β, γ ratio of genes or expression produces as 2:1:1 or other ratios where the Aα gene predominates instead of the presently used ratio 1:1:1.

In a preliminary testing it appeared that the β chain may be the limiting factor, at least for the outcome related to the total yield of recombinant fibrinogen, using 1:2:1 or even higher predomincance of the β gene, e.g. as much as 1:5:1 (however, the initial findings seemed to point at the direction that 1:5:1 would be relatively toxic for the HEK 293t cell culture). So also here, a ration between α, β, and γ chains different from 1:1:1 with a predominance of the β chain is preferred. In both types of cases, the obtained F5 and F6 polypeptides may strengthen or provide polymerization when induced or even when mixed with Bβ and with γ chain proteins. Thus it is within the scope of this invention when F5 and F6 fibrinogens are provided in parts or as totally intact fibrinogen molecules.

This invention also covers the use of a new and improved promoter, and vector system, and a new HEK cell line (the promoter and vector and HEK cell line from HumanZyme Inc., Chicago, Ill.) for the production of the F5 and F6 fibrinogen molecules.

Bβ gene(s) resulting in an amino acid correction in at the amino acid sequence of the Bβ chain may be involved in optimizing the fibrinogen composition, which may be most useful under various conditions, such as for instance, when highly reliable fibrinogen is used for systemic treatment of patients with various fibrinogen abnormities, such as dysfibrinogenemia, hypofibrinogenemia, as well as a possible life saving fibrinogen as an essential treatment of severely bleeding trauma patients ranging from serious accidents, disasters, combat and explosives related situations. The original human fibrinogen α, β, and γ genes used for preparing the recombinant fibrinogen, were purchased—the relevant accession Nos. are: NM_{—}021871 for Fb_Aα; BC106760 for Fb_Bβ and BC021674 for Fb_γ. These genes were tested using PCR for fibrinogen α, β, γ genes. When we analyzed the purchased BC106760 fpr FB_Bβ gene, it appeared to have a proline at aa#162 in the Bβ fibrinogen protein chain. We then changed the proline at this position 162 to alanine in order to prepare the human wild-type fibrinogen. It is still not completely established whether the human fibrinogen beta chain in nature appears with a proline or an alanine in position 162, but from the point of view that the most optimal plasma fibrinogen standard, which has been tested (Calbiochem human plasma fibrinogen), the recombinant human fibrinogen with proline at position 162 showed a polymerization at a log higher amount of this recombinant fibrinogen, when compared to the current recombinant human fibrinogen, the version with alanine in position 162 of the beta chain is highly preferred according to this invention.

However, other variants focusing on amino acid changes in position 162 of the beta chain, which is contemplated to exert improved polymerization characteristics. Thus, any non-proline L-amino acid residue in position 162 of the beta chain is a mutant or variant of relevance in the present invention as is fibrinogens having such a mutant/variant beta chain. Thus, the amino acid in position 162 is e.g. selected from L-alanine, L-cysteine, L-aspartic acid, L-glutamic acid, L-phenylalanine, glycine, L-histidine, L-isoleucine, L-lysine, L-leucine, L-methionine, L-asparagine, L-glutamine, L-arginine, L-serine, L-threonine, L-valine, L-tryptophan, and L-tyrosine.

It is also anticipated and within the scope of this invention that F5 recombinant fibrinogen or more specifically F5 mammal recombinant fibrinogen or even more specifically, F5 human recombinant fibrinogen could to a high degree be one of the plasma fibrinogens normally harvested or predominantly one of the plasma fibrinogens harvested or possibly constituting the major plasma fibrinogen resembling F5. Thus, the F6 fibrinogen may still be present in many mammals including man, but there might be a difference in the relative concentration of a plasma derived F5 fibrinogen and of plasma derived F6 fibrinogen. The polymerization curve of plasma fibrinogen resembles to a high degree the polymerization curve of F5 recombinant human fibrinogen found in this invention. So, if F5 and F6 as expected appear in mixed form in plasma with the highest relative concentration of the plasma F5 component or molecule, the polymerization curve would or might tend to be close to the purified and isolated F5 recombinant human fibrinogen, cf. the observation reported herein that when mixing F5 recombinant human fibrinogen and F6 recombinant fibrinogen at a ratio of 50:50 the polymerization curve tends to be relatively closer to F5 than F6.

### METHOD OF USE

The method of the invention and the preparation of the invention provides for fibrinogen, which will find use in the same settings as would normally be relevant for plasma fibrinogen or other state-of-the-art fibrinogen preparations. One of these settings is prevention or treatment of bleeding, especially of excessive bleeding.

According to this invention the three individual fibrinogen encoding genes are e.g. transfected into cells, especially and primarily those that are of human origin, in order to obtain a human glycosylation pattern of the resulting particular fibrinogens such as for instance F5 and F6, whichever of these may be most suitable for systemic treatment for instance by injection or infusion into humans or mammals.

Diseases or conditions that may be targeted by administration and activation of the fibrinogens of the present invention are those normally subject to treatment with fibrin/fibrinogen: severe bleedings from various conditions ranging from medical conditions characterized by altered or lowered fibrinogen concentration or activity as well as trauma inflicted by surgical procedures or by accident/injury. Further, excessive consumption by fibrinogen may also lead to a need for exogeneous administration. Also in transplantation applications and for topical use, the fibrinogens of the present invention will find use. For instance when creating a recombinant fibrin glue or recombinant tissue sealant for the treatment of topical bleedings for instance during surgery or other intervention, the fibrinogens of the present invention will be useful.

Hence, in a third aspect the invention provides a method of treatment or prophylaxis of bleeding in an individual, the method comprising administering an effective amount of a preparation of the second aspect of the invention or of a fibrinogen obtained by the method constituting the first aspect of the invention.

For instance, wherein administering fibrinogen can be effective in substantially maintaining or restoring blood clotting capability in disrupted/traumatized vasculature. This can have practical implications in surgical methods, in first-aid settings or damage control settings (such as when offering first-line treatment to individuals which have been severely physically traumatized, as may be the case for combat soldiers, victims of accidents or terrorism), or in any other situation where the recipient of the fibrinogen is suffering from or is likely to start suffering from excessive bleeding. For instance: when the individual, prior to said treatment or prophylaxis, suffers from or is susceptible to bleeding as a consequence of a dysfunction or lack of functioning fibrinogen, an injury, past or future surgical treatment (where heavy bleeding is expected; one example being heart surgery where about 5% such as bypass surgery; or in order to join tissue such as vessels, organs, brain tissue, and join bridging tissue defects to repair said defects), post-partum hemmorhage, dissiminated intravascular coagulation (where the patient's fibrinogen level is lowered due to disseminated clotting resulting in development of fibrin split products, which are produced within fibrin and which undergoes degradation when blood clots are dissolved, and where it can be necessary to treat the patient systemically with fibrinogen), and organ transplantation (such a liver transplantation or other organ transplantations where the plasma fibrinogen will be lowered).

Administration of fibrinogen according to the invention may be made both systemically or locally, depending on the circumstances—typically systemic administration is preferred when the individual has a general loss of autologous fibrinogen due to the above indicated general circumstances or conditions, whereas local or topical administration is normally preferred under more controlled settings—in that case, the fibrinogen will often be administered together with thrombin or an analogue thereof (the latter term indicating a molecule having the same biologic activity as thrombin with respect to activation of fibrinogen into fibrin—an analogue of thrombin may e.g. be a human thrombin mutant, such as a point mutated version or a biologically active thrombin fragment; cf. also WO 2007/103447 where suitable thrombin molecules and their preparation is provided).

One such “controlled setting” is the case wherein thrombin or the analogue thereof is administered together with fibrinogen in order to glue organs or cells together, as may be the case when performing surgery on organs where normal surgical ligations are less effective (but generally in organs or various vessels such as blood vessels, urinary tract vessels, urogenital vessels, intestines and other hollow organs). Also, thrombin or the analogue therefore is administered together with fibrinogen in order to effect hemostasis during surgery or in settings where local bleeding is excessive but where there is not need or possibility of administering systemic fibrinogen. Also a method of ligating a surgical wound in an individual where fibrinogen is administered, optionally in combination with thrombin or an analogue thereof, is a part of the third aspect.

The invention also entails transplanting cells or tissue to an individual, the method comprising administering an effective amount of a preparation according to the second aspect of the invention or a fibrinogen obtained by the method of the first aspect of the invention to said individual, optionally in combination with administration of cells and/or tissue factors and/or thrombin and/or an analogue of thrombin. Here, the thrombin is essentially added when necessary (e.g. when the body of the individual does not provide sufficient thrombin activity at the site of transplantation). Tissue factor and cells are provided as and when conventional in the particular type of transplantation—when transplanting cells in order to regenerate tissue, it is known that further addition of local tissue growth factors may be of relevance, as may the addition of cells of connective tissue origin in order to facilitate the result of the transplantation.

Related to this, the invention also relates to a method of adhering cells such as stem cells or progenitor cells or precursor cells to a target location in an individual in which said cells shall adhere in order to function adequately for the repair of neighbouring cells and tissue, the method comprising administering fibrinogen according to the invention, optionally in combination with administration of thrombin or a thrombin analogue—again, the thrombin/thrombin analogue is administered as necessary and according to the normal use in the art.

Preferred embodiments of the third aspect of the invention are those, where the individual is a human being.

From a polymerization standpoint, the F6 recombinant fibrinogen is preferred for treatment of patients, especially those patients who are severely traumatized such as for instance soldiers wounded in combat, where multiple bleeding locations on the patient shows a rapid loss of fibrinogen to a level, where clotting attempts is almost impossible. Due to our findings that F6 shows a very satisfactory fast polymerization, and due the fact that F6 fibrinogen is much more stable (does not undergo degredation as fast as F5), this enables the skilled person to prepare F6 fibrinogen (e.g. in dried, such as freeze-dried, form), which can be dissolved in solvents brought directly to the trauma site, e.g., in the combat field, and after the F6 has been dissolved, it could be readily infused systemically and thereby bring the fibrinogen level up and thereby minimize otherwise excessive bleeding.

Apart from the above-discussed kinds of trauma, postpartum hemorrhage and heart bypass surgery has proven to cause excessive and dangerous bleedings (for instance in by pass the postoperative bleedings caused by fibrinogen depletion demanding surgical re-intervention is around 5%)—so also in these settings, the thirds aspect of the invention is relevant. In both trauma and in these disease related or post-surgical related bleedings, it is of major importance to have access to a fibrinogen such as for instance F6, which exhibits a slow degradation of the protein compared to the major part of fibrinogen products currently available.

If one should compensate for at profuse bleeding of say 2-3 liters within 15 to 30 minutes, the amount of cryoprecipate units of traditional fibrinogen that should be administered would in most cases not be practically possible, because one had to most probably infuse 15 to 25 units of cryoprecipitate. In such cases, a preparation of F6 fibrinogen according to the present invention, which does not contain non active ingredients or even counterreactive agents such as fibronectins, would provide for an excellent alternative.

Finally, when performing cell transplantation whether autologous, allogenic, or xenogenic the combination of the fibrinogens of the present invention with human or mammal thrombin such as recombinant (human) mammal thrombin of wild type or M84A mutants disclosed in WO 2007/103447 is within the scope of this invention to facilitate the grafting of such cells, as for instance when performing cartilage repair, bone repair, cardiac cell repair or muscle repair.

With respect to dosage, this will in cases of systemic administration entail administration of amounts sufficient to establish a serum concentration of fibrinogen, which will allow blood-clotting—in other words, the amount to be administered will depend on the severity of the condition treated; heavy bleeding will require more fibrinogen than smaller bleedings, etc. It is preferred that the amount administered will be sufficient to reestablish a serum concentration of fibrinogen which is considered within or above the normal serum concentration.

A fourth aspect of the present invention relates to a method of adhering cells to a solid or semi-solid surface by contacting said cells and said solid or semi-solid surface with a preparation according to the second aspect of the invention or with a fibrinogen obtained by the method of the first aspect of the invention.

It will from the above be understood that the invention pertains to a preparation of the second aspect of the invention or a fibrinogen obtained by the method of the first aspect of the invention for use as a pharmaceutical. In particular the invention relates to a preparation of the second aspect of the invention or a fibrinogen obtained by the method of the first aspect of the invention use in a method according to the third aspect of the invention.

### KITS OF THE INVENTION

The invention also enables the provision of kits suitable for exercising the 3^{rd} and 4^{th} aspects of the invention. For instance, the invention relates to a kit comprising, in separate containers or vessels, 1) a preparation of the second aspect of the invention or a fibrinogen obtained by the method of the first aspect of the invention, and 2) thrombin or an analogue thereof, and optionally 3) Factor XIII (cf. below). It is particularly preferred that at least one of each (preferably all) of the protein components of the kit are provided in dry form in the containers/vessels, in particular in freeze-dried form as this enables increased stability, ease of transport and ease of admixing the constituents with a liquid to provide a composition suitable for injection or topical/local administration.

### OTHER ISSUES/USES

It is contemplated that the present observation that the same fibrinogen encoding genes result in provision of multi-PT species fibrinogens is also applicable to other animal fibrinogens. Hence, it is within the scope of the invention to clone fibrinogen encoding genes of any mammal into a suitable expression vector and to subsequently transfect suitable host cells (preferably of the same species origin as the fibrinogen genes) and effect expression of these genes to obtain at least two PT species of fibrinogen. Likewise, it is also within the scope of this invention to clone an animal, such as a mammal, harbouring a cell clone which may be capable also of producing the two PT species such as F5 and F6, whether similar to human or almost similar to human fibrinogen. So irrespectively of how the F5 and F6 fibrinogen can be produced individually or in pairs is considered part of the present invention.

It is expected based on the present findings, that at least two PT species of fibrinogen, e.g. corresponding to recombinant fibrinogen F5 and F6, may be produced also in cells such as CHO cells although such multi PT species have not hitherto been identified. It is indeed according to this invention by careful examination of PAGE gels highly likely that at least two PT fibrinogen species are also present in mammal plasma, such as in human plasma. However, until now, it has not been possible to discriminate and e.g. prepare isolated preparations of F5 and F6 (or other separate PT species). Thus, with the present invention it has for the first time become possible to prepare compositions having defined relative concentrations of each the existing PT fibrinogen species suitable for specific fibrinogen products.

In this invention we have primarily focused on producing the F5 and F6 species in HEK cells, in particular the HEK293t and HEK293ts cells licensed from HumanZyme, Chicago, Ill. Other human cell lines are however considered useful for the same purpose, such as for instance PER cells, in particular PER 6 and PER C6 cells.

According to this invention two promoter and vector systems licensed from HumanZyme have proven particularly suitable for the transfection into human cells and suitable for expressing the fibrinogens when using human cell systems; however the invention is not limited to use of these promoter and vector systems, but may be substituted by other commercially available or experimental promoter and vector systems known the person skilled in the art.

Activation of the fibrinogens and fibrinogen mixtures of the present invention may be achieved by either plasma thrombin or recombinant mammal thrombin or more specifically, recombinant human thrombin, e.g. recombinant human authentic-like thrombin having the thrombin wild-type sequence or the M84A mutant recombinant human thrombin sequence, cf. WO 2007/103447.

Also FXIII may play a role in the rate by which fibrinogen may be clotting, when cleaved by thrombin, however, in regards to the scope of this invention, FXIII is not taken into consideration when testing was performed on recombinant human fibrinogen F5 and/or F6. On the other hand the present inventor is taking into consideration, the possible influence of FXIII including the influence related to possible changes in FXIII molecule might further aid to the individual polymerization and or clotting effects of F5 and F6 fibrinogens. Hence, according to the present invention, exact choices of other factors and chemical agents that affect activation of fibrinogen into fibrin may be adjusted and taken into consideration when putting the present invention into practice. However, according to the present invention, all embodiments of the methods of the third and fourth aspects of the invention may be combined with administration of FXIII in order to improve the effect of the fibrinogen administered.

### LEGEND TO THE FIGURES

FIG. 1 shows elution curves from IMAC of recombinantly produced fibrinogen 2 protein peaks each containing recombinant human fibrinogen made from the HEK293t cell. The 2 peaks were eluted by application of 20 mM L-arginine as well as 50 mM arginine to yield the F5 and F6 proteins respectively.

FIG. 2 shows further fractionation on a heparin column eluted with TBS buffer of the material from the protein peaks in FIG. 1.

FIG. 2A: The F5 recombinant human fibrinogen fraction obtained on the IMAC column at 20 mM L-arginine appeared without major tailing.

FIG. 2B: The F6 recombinant human fibrinogen fraction obtained on the IMAC column at 50 mM L-arginine appeared with some tailing of the protein.

FIG. 3 shows the results of PAGE gel electrophoresis performed on the F5 and F6 proteins after IMAC and heparin column purification.

Results are shown for both non-reduced (marked with “−”) and reduced (marked with (“+”) protein. It is observed that the Aα band in reduced F5 is broader and somewhat diffuse, and not as distinct when compared to F6. The reduced Bβ and the γ bands in both F5 and F6 are virtually of the same intensity.

FIG. 4 shows PAGE gel results for purified fibrinogens.

Lanes on the left side, #1 through 4, show purified fibrinogens in non-reduced conditions, and the right side PAGE gel Lanes #1 through 4 show reduced fibrinogens. Lane 1 non-reduced gel shows plasma fibrinogen (Sigma #F4883), with a broader band resembling the non-reduced band in Lane 4, which is the F5 recombinant human fibrinogen from HEK cells, Lane 2 shows non-reduced recombinant fibrinogen from CHO cells, a broader and more diffuse band, which is located lower than the other non-reduced fibrinogens; lane 3 shows non-reduced F6 recombinant human fibrinogen from the HEK cells that is more dense than those of the other fibrinogens on the gel, and lane 4 shows non-reduced F5 recombinant human fibrinogen from HEK cells, as described resembling the non-reduced plasma fibrinogen in lane 1. The non-reduced lane 5 shows the mixture of F6 and F5, where the band is more intense most probably due to the influence of F6.

On the reduced PAGE gel, Lane 1, the Aα chain resembles to some degree the Aα in Lane 4 (F5 recombinant human fibrinogen) being more diffuse and weaker than the other Aα chains from the other fibrinogens. Bβ and γ chains in Lane 1 resemble the same bands in Lane 4. The Aα chain of the recombinant fibrinogen from CHO cells in Lane 2 is slim and weaker than the Aα chains of the other fibrinogens; the Bβ chain of the recombinant fibrinogen from CHO cells is located somewhat lower than the Bβ chain of the other fibrinogens. The reduced Lane 5 shows the mixture of F6 and F5, where the Aα band is more intense most probably due to the influence of F6.

FIG. 5 shows polymerization testing of fibrinogens.

The fibrinogens were tested at a concentration of 0.2 mg/ml, induced by thrombin 1 μg/ml in TBS with 10 mM CaCl₂ to polymerization. It is apparent that F5 recombinant human fibrinogen made in HEK cells according to the invention shows the same polymerization curve as plasma fibrinogen (Sigma F4883), whereas F6 recombinant human fibrinogen made in HEK cells according to this invention follows the polymerization curve as shown by recombinant fibrinogen made in CHO cells.

FIG. 6 shows polymerization testing of fibrinogens.

The fibrinogens were tested at a concentration of 0.2 mg/ml, induced by thrombin 1 μg/ml in TBS with 10 mM CaCl2 and polymerized under these conditions. It is apparent that F5 recombinant human fibrinogen exhibits substantially the same polymerization curve as plasma fibrinogen (Sigma F4883), whereas F6 recombinant human fibrinogen shows a significantly different polymerization pattern when compared to recombinant human fibrinogen F5 and to plasma fibrinogen (F4883). When mixing (50/50) F5 and F6 the polymerization curve approaches F5 more than would be expected at a 50/50 mix.

### PREAMBLE TO EXAMPLES

The gene material used for preparing the Aα, Bβ and γ pair of fibrinogen genes in the following examples has previously been described: The original human fibrinogen Aα, Bβ, and γ genes used for preparing the recombinant fibrinogen were purchased as GB accession No.: NM_{—}021871 for Fb_Aα; BC106760 for Fb_Bβ and BC021674 for Fb_γ, with the exception that the accession number BC106760 for the Bβ chain with a base pair size of 156 at Primer 5′-3′ sequence SEQ ID NO: 3: GTGAATAGCAATATCC (for the expression of proline at position No. 162) was changed to a Bβ encoding chain with a base pair size of 156 at Primer 5′-3′ sequence SEQ ID NO: 4: GTGAATAGGCAATATCG encoding alanine in position 162. As described above, it is not completely established in the current accessible gene material whether the Bβ containing proline or containing alanine is the correct wild type, however the most active form of these two different fibrinogens most probably is the true wild type—at any rate, the most active form is the preferred one according to the present invention.

The resulting Bβ fibrinogen amino acid sequence appears from SEQ ID NO: 2, whereas the sequence originally encoded by BC106760 appears from SEQ ID NO: 1.

### Example 1

HEK 293t cells (HumanZyme, and licensed from HumanZyme) as well as several variants of the HEK cell was tested for the transfection of the Aα, Bβ, and γ fibrinogen genes built into the PHZsec vector—promoter system (licensed from HumanZyme).

The vectors were propagated in E. coli, a multiple vector/gene pool was obtained, subsequently transfected into the various HEK cells and cultured in serum containing medium as a monolayer cell line. Clones were selected and grown separately. The clones producing fibrinogen (with alanine in amino acid position 162 in the beta chain) were selected. The optimally producing cell clones were selected and chosen for adaptation to serum free medium (HumanZyme, licensed from HumanZyme) or serum free, chemically defined medium (HumanZyme, licensed from HumanZyme) during approximately one week in order to be adapted into a cell suspension culture.

The previously used HEK293cell line used and described in WO 2007/103447 appeared to not being capable of being transferred to suspension culture, but continued to clot or clump together. Another cell line (the HEK293t cell line from HumanZyme, licensed from HumanZyme) cell was chosen and appeared to be capable of adapting to a suspension culture, and proved to produce recombinant human fibrinogen. This transformed cell line was hence processed into a suspension culture and the fibrinogen was isolated.

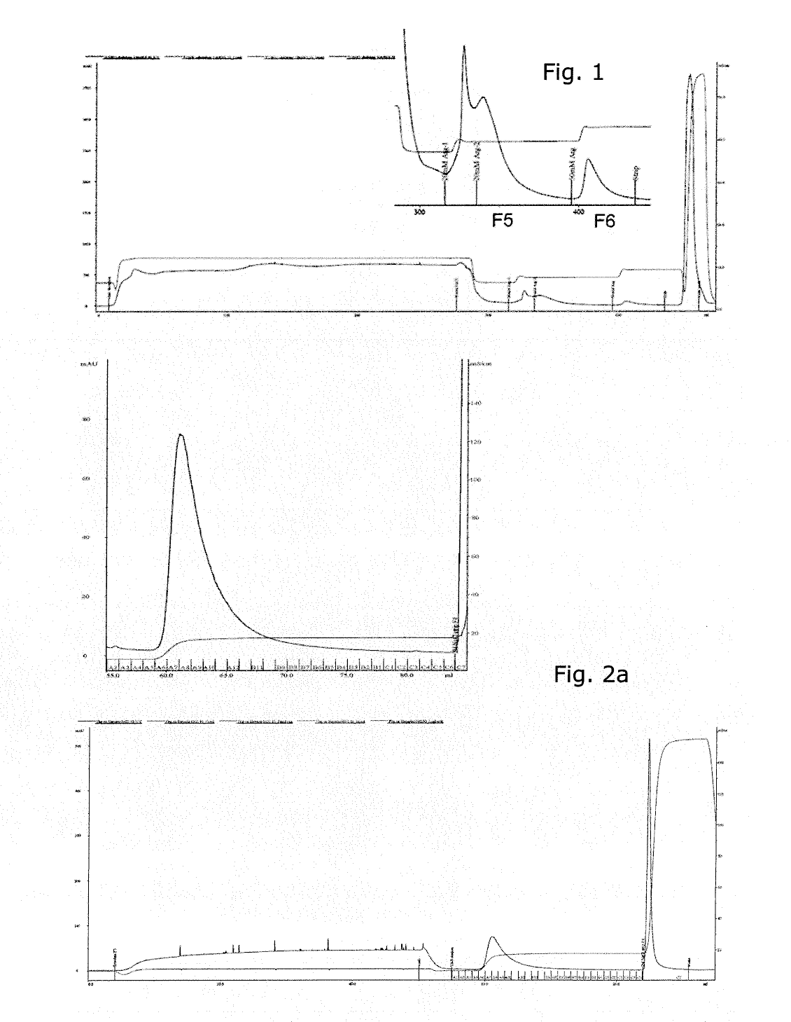

Surprisingly, two different fibrinogen bands could be purified on the IMAC (immobilized metal affinity chromatography) column as shown in FIG. 1. It appeared that one significant protein peak, containing recombinant human fibrinogen, appeared when 20 mM L-arginine was added during the IMAC procedure; when the L-arginine concentration was elevated to 50 mM during the IMAC procedure, a second significant protein peak also containing recombinant human fibrinogen appeared clearly.

It was found that the protein peak representing the recombinant human fibrinogen appearing at 20 mM L-arginine was significantly different from the recombinant human fibrinogen appearing at 50 mM L-arginine in regards to its polymerization characteristics as well as its appearance on a PAGE gel; the fibrinogen appearing at 20 mM L-arginine was named “F5 recombinant human fibrinogen” (or just “F5”).

The second protein peak, appearing at 50 mM L-arginine and also containing recombinant human fibrinogen, appeared to differ significantly from the F5 recombinant human fibrinogen in regards to its polymerization and its appearance described in studies described below. The fibrinogen appearing in this second peak was named “F6 recombinant human fibrinogen” or just “F6”.

### Example 2

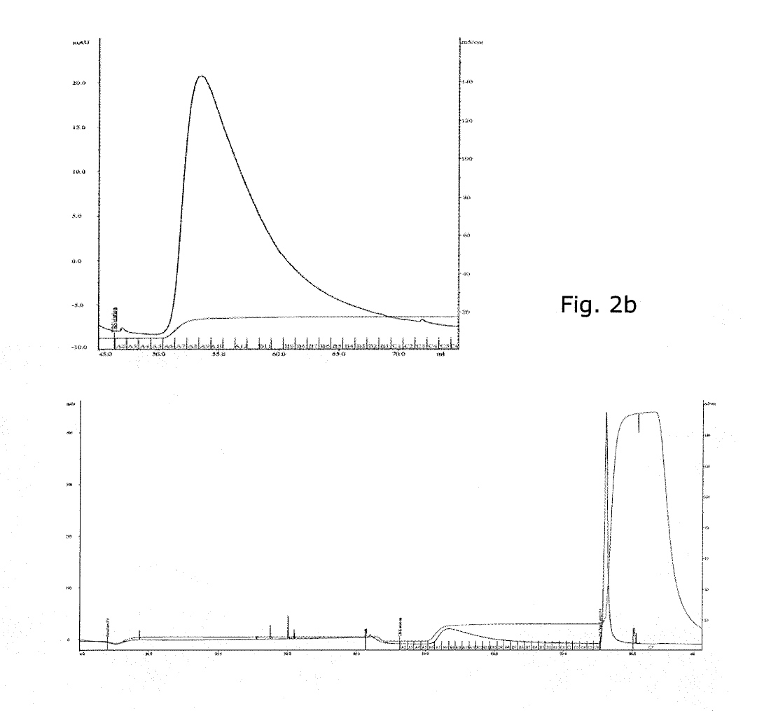

The fractions containing the significant F5 protein peak as demonstrated in Example 1 and FIG. 1 were pooled and applied to a heparin column and eluted with TBS buffer. The resulting purification on the heparin column is shown in FIG. 2A.

The fractions containing the significant F6 protein peak as demonstrated in Example 1 and FIG. 1 were pooled and was then applied to a heparin column and eluted with TBS buffer. The resulting purification on the heparin column is shown in FIG. 2B.

### Example 3

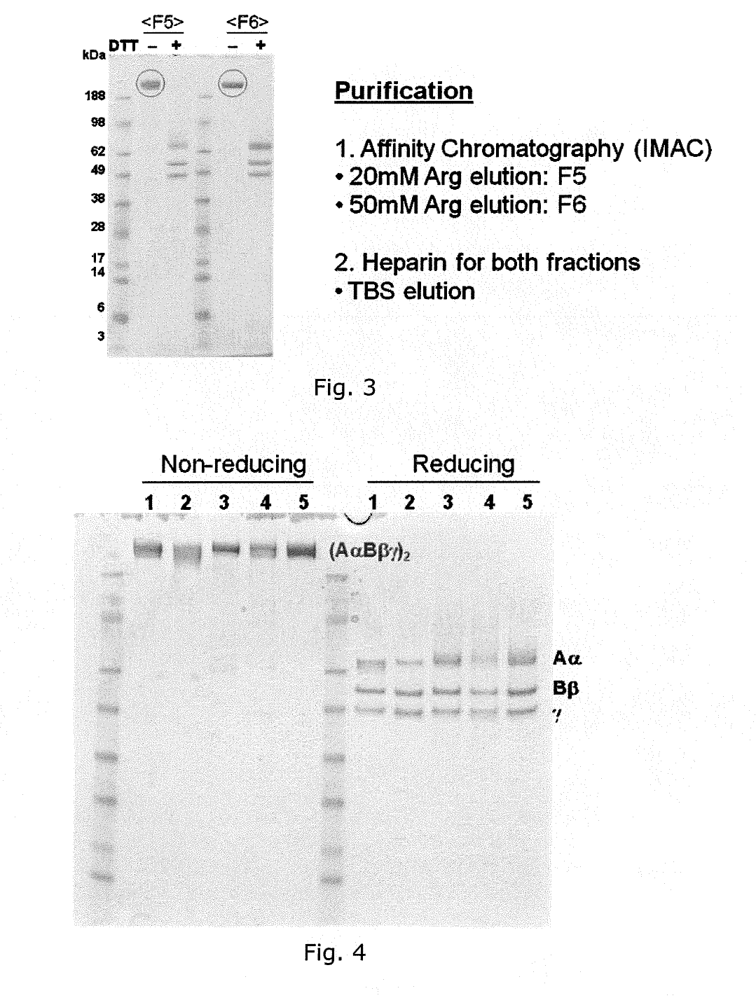

The purified F5 and F6 recombinant human fibrinogens were tested individually by PAGE gel electrophoresis as shown in FIG. 3. It appears that the non-reduced F5 recombinant human fibrinogen on the gel shows a broader band than observed for F6 where the non-reduced band appeared more distinct, and less fuzzy.

When F5 was reduced, the “F5 Aα”, appeared more diffuse than the “F6 Aα, which appeared more distinct and higher in relative concentration whereas there is less difference between “F5 Bβ” and “F6 Bβ” as well as between “F5 γ” and “F6 γ” bands.

### Example 4

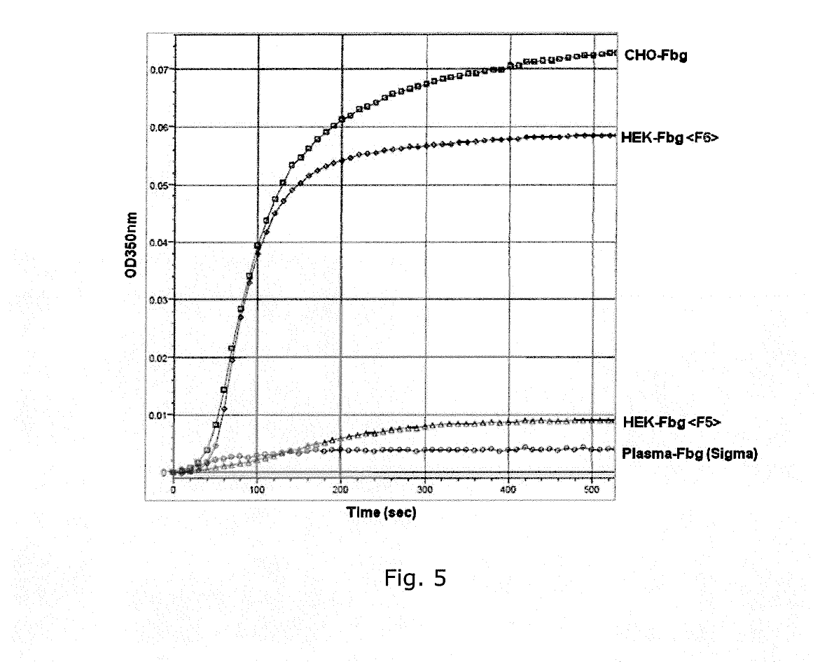

The following comparison of fibrinogens was made using PAGE gel electrophoresis. In the lanes on the left side of FIG. 4 are shown non-reduced fibrinogen proteins and on the right side reduced fibrinogen proteins.

In left and right lanes 1 non-reduced and reduced plasma fibrinogen (Sigma F4883) was applied. In left and right lanes 2, non-reduced and reduced recombinant fibrinogen made from CHO cells are shown. In left and right lanes 3, non-reduced and reduced F6 recombinant human fibrinogen is shown, and in left and right lanes 4, non-reduced and reduced F5 recombinant human fibrinogen is shown.

It appears that in the non-reduced lanes, plasma fibrinogen (Sigma F4883) resembles non-reduced F5 recombinant human fibrinogen. This interesting observation should be compared with what is revealed in Example 5 regarding polymerization. In lane 3 non-reduced F6 recombinant human fibrinogen from HEK cells is different from the other non-reduced fibrinogens because the band is narrow and distinct. Lane 5 is a 50/50 mixture of F6- and F5 recombinant human fibrinogens. The non-reduced lane 5 shows the mixture of F6 and F5 (50/50), where the band is more intense, most probably due to the influence of F6 (FIG. 4).

In the reduced lane 1, the Aα for plasma fibrinogen and in lane 4 the reduced Aα chain for recombinant fibrinogen F5 does not appear to show any significant difference, although close inspection of the gel does seem to indicate that reduced lane 1 contains 2 separate Aα bands (one diffuse and a more distinct just above the diffuse band), whereas reduced lane 4 only contains one fussy band; this could imply, that the plasma fibrinogen in reality contains to distinct Aα species. The reduced Bβ chain for plasma fibrinogen is resembling the reduced Bβ chain for F5 recombinant human fibrinogen, and reduced γ chain for plasma fibrinogen also to some degree resembles reduced γ chain for F5 recombinant human fibrinogen.

The position of the non-reduced recombinant fibrinogen from CHO cells in lane 2 is located significantly lower and is relatively more diffuse and broader than any of the other non-reduced fibrinogens. This is most likely due to the fact that CHO cell is a hamster derived cell, which provides for a different glycosylation pattern when compared to recombinant human fibrinogens made in human cells—glycosylation patterns are decided by the nature of the transfected cell rather than by the gene that was transfected into the cell, or in other words, not decided by the gene inserted in the particular cell but due to post-translational modification (PTM). Hence, the choice of host cell may alter the resulting processed and secreted glycoproteins in regards to their physical and chemical properties, e.g., MW, pI, folding, stability, and biological activity. Also the phosphorylation is perceived to be decided by post-translational modification.

The reduced lane 3 shows the F6 recombinant human fibrinogen from HEK cells, which shows a considerably stronger and more dense Aα band than observed in any of the other fibrinogens (FIG. 4).

When viewing the reduced lane 3 (F6), it is interesting that the reduced Aα chain of recombinant human fibrinogen from human cells is a very slim and condensed band, when compared to the reduced Aα chains from the other fibrinogens. It also appears that the reduced Bβ and γ chains from the recombinant fibrinogen from CHO cells are located lower than the reduced Bβ and γ chains for the other fibrinogens. So, it appears that the recombinant fibrinogen differs based on the location of the bands both under non-reducing conditions as well under reducing conditions. Lane 4 representing the non-reduced F6 recombinant human fibrinogen from human cells, differs from the other non-reduced fibrinogens because as described, the Aα band is more dense and less spread out, when compared to the other non-reduced fibrinogens. Reduced Lane 5 shows the mixture of F6 and F5, where the Aα band is more intense most probably due to the influence of F6 (FIG. 4).

### Example 5

The polymerization of plasma fibrinogen (Sigma F4883) was compared to the polymerization of recombinant fibrinogen from CHO cells, F5 recombinant human fibrinogen, and F6 recombinant human fibrinogen both made in transfected HEK cells according to this invention. In this assay Fibrinogens were added at a concentration of 0.2 mg/mL and induced with thrombin 1 μg/mL in TBS with 10 mM CaCl₂.

It is evident from this study that F5 recombinant human fibrinogen shows essentially the same polymerization curve as plasma fibrinogen, whereas F6 recombinant human fibrinogen shows a different polymerization curve. The F6 polymerization curve resembles to a certain degree the polymerization curve shown by the recombinant fibrinogen from CHO, cf. FIG. 5.

### Example 6

The polymerization of plasma fibrinogen (Sigma F4883) was compared to the polymerization of recombinant human fibrinogen F5, recombinant human fibrinogen F6, and a 50/50 mixture of F5 recombinant human fibrinogen and F6 recombinant human fibrinogen. In this assay fibrinogen was added at a concentration of 0.2 mg/mL and induced with thrombin 1 μg/mL in TBS with 10 mM CaCl₂.

It appears again that recombinant human fibrinogen F5 shows a polymerization curve at the same level as plasma fibrinogen (Sigma F4883). The recombinant human fibrinogen F6 appears again to differ significantly from recombinant human fibrinogen F5 and from plasma fibrinogen (Sigma F4883).

When inspecting the start of the polymerization under these conditions, all fibrinogens tested seem to polymerize at about the same time, with a minor non-significant difference. When F5 and F6 were mixed 50/50, it was expected that the polymerization would show a curve almost between F5 and F6 level, but instead the F5/F6 mix surprisingly turned out to be much closer to F5 and plasma fibrinogen.

This observation points in the direction that even under a theoretical mix of a possible plasma fibrinogen F5 and a possible plasma fibrinogen F6, the polymerization characteristics may be dominated by fibrinogen F5 rather than by F6, which might speak against a possible deterioration of F6 in plasma either due to a less stable fibrinogen F6 and also speaks against a possible inactivation during heat treatment of plasma fibrinogen of F6, due to the fact that the two recombinant human fibrinogens F5 and F6 were kept under identical conditions before and during the entire testing. Actually, the recombinant fibrinogens F5 and F6 were kept at −80° C. until polymerization testing.

1. A method for preparing fibrinogen comprising:
providing cells transfected with expression vectors encoding individual fibrinogen chains, subsequently culturing said cells under conditions that facilitate production by said cells of fibrinogen, and subsequently recovering said fibrinogen and separating said fibrinogen in at least two post-translationally modified species distinguished by differing migration patterns in a PAGE gel and/or by differing polymerization upon activation with thrombin.

2. The method according to claim 1, wherein the fibrinogen is human fibrinogen or a human fibrinogen including up to 10 amino acid mutations in each fibrinogen chain.

3. The method according to claim 2, wherein the transfected cells are constituted by human cells, selected from a human cell clone or human cell line.

4. The method according to claim 4, wherein the transfected cells are HEK cells, such as HEK293t and HEK293ts cells.

5. The method according to any one of claims 2-4, wherein the human fibrinogen is separated into at least two post-translationally modified forms, wherein one (F5) has a PAGE migration pattern and polymerization characteristics similar to serum fibrinogen and wherein another (F6) has a PAGE migration pattern, which differs in band intensity, but has polymerization characteristics similar to fibrinogen expressed by CHO cells.

6. A substantially pure preparation of human fibrinogen, which is free from non-fibrinogen serum protein and where the fibrinogen consists of:
a) F5 fibrinogen,
b) F6 fibrinogen, or
c) a mixture of F5 and F6 fibrinogen.

7. The substantially pure preparation according to claim 6, wherein the fibrinogen is native human fibrinogen or a human fibrinogen including up to 10 amino acid mutations in each fibrinogen chain.

8. The substantially pure preparation according to claim 7, wherein the human fibrinogen includes a non-proline as amino acid residue 162 in the β-chain, such as an alanine residue.

9. A method of treatment or prophylaxis of bleeding in an individual, the method comprising administering an effective amount of a preparation according to any one of claims 6-8 or a fibrinogen obtained by the method of any one of claims 1-5 to said individual.

10. The method according to claim 9, wherein administering said preparation according to any one of claims 6-8 or a fibrinogen obtained by the method of any one of claims 1-5 is effective in substantially maintaining or restoring blood clotting capability in disrupted/traumatized vasculature.

11. The method according to claim 9 or 10, wherein said individual, prior to said treatment or prophylaxis, suffers from or is susceptible to bleeding as a consequence of a dysfunction or lack of functioning fibrinogen, an injury, past or future surgical treatment, post-partum hemmorhage, dissiminated intravascular coagulation, and organ transplantation.

12. The method according to claim 11, wherein administering an effective amount of a preparation according to any one of claims 6-8 or a fibrinogen obtained by the method of any one of claims 1-5 is made systemically.

13. The method according to claim 11, wherein administering an effective amount of a preparation according to any one of claims 6-8 or a fibrinogen obtained by the method of any one of claims 1-5 is made topically or locally, optionally in combination with thrombin or an analogue thereof.

14. The method according to claim 13, wherein the preparation or fibrinogen, the thrombin or the analogue therefore is administered in order to glue organs or cells together.

15. The method according to claim 13, wherein the preparation or fibrinogen, the thrombin or the analogue thereof is administered in order to effect hemostasis.

16. A method of transplanting cells or tissue to an individual, the method comprising administering an effective amount of a preparation according to any one of claims 6-8 or a fibrinogen obtained by the method of any one of claims 1-5 to said individual, optionally in combination with administration of cells and/or tissue factors and/or thrombin and/or an analogue of thrombin is also administered.

17. A method of adhering cells such as stem cells or progenitor cells or precursor cells to a target location in an individual in which said cells shall adhere in order to function adequately for the repair of neighbouring cells and tissue, the method comprising administering an effective amount of a preparation according to any one of claims 6-8 or a fibrinogen obtained by the method of any one of claims 1-5 to said individual, optionally in combination with administration of thrombin or a thrombin analogue.

18. A method of ligating a surgical wound in an individual, the method comprising administering an effective amount of a preparation according to any one of claims 6-8 or a fibrinogen obtained by the method of any one of claims 1-5 to said individual, optionally in combination with thrombin or an analogue thereof.

19. The method according to any one of claims 9-18, wherein the individual is a human being.

20. A method of adhering cells to a solid or semi-solid surface by contacting said cells and said solid or semi-solid surface with a preparation according to any one of claims 6-8 or a fibrinogen obtained by the method of any one of claims 1-5.

21. A kit comprising, in separate containers or vessels, 1) a preparation according to any one of claims 6-8 or a fibrinogen obtained by the method of any one of claims 1-5, and 2) thrombin or an analogue thereof, and optionally 3) Factor XIII.

22. The kit according to claim 21, wherein 1) the preparation according to any one of claims 6-8 or a fibrinogen obtained by the method of any one of claims 1-5 and/or 2) the thrombin or an analogue thereof and/or 3) Factor XIII is/are dried such as freeze dried.

23. A preparation according to any one of claims 6-8 or a fibrinogen obtained by the method of any one of claims 1-5 for use as a pharmaceutical.

24. A preparation according to any one of claims 6-8 or a fibrinogen obtained by the method of any one of claims 1-5, for use in a method according to any one of claims 9-19.
